SEQUENCE LISTING (2 sequences)

SEQ ID NO: 1 - DNA, 47 bp, Artificial Sequence
  PCR primer
ggaattccatatgcaccaccaccaccaccacgcagagcagagagcca

SEQ ID NO: 2 - DNA, 31 bp, Artificial Sequence
  PCR primer
catgccatggcctccactaactcagtttcct

(19) United States
(12) Patent Application Publication
(10) Pub. No.: US 2004/0142429 A1
(43) Pub. Date: Jul. 22, 2004

(54) Method for the expression, purification, and structure recovery and handling of hydrophobic proteins, polypeptides or peptides

(76) Inventors: Chris W.M. Grant, London (CA); David Jones, San Diego, CA (US)

Correspondence Address: OGILVY RENAULT, 1981 MCGILL COLLEGE AVENUE, SUITE 1600, MONTREAL, QC, H3A2Y3, CA

(21) Appl. No.: 09/768,313

(22) Filed: Jan. 25, 2001

(51) Int. Cl. C12P 21/04; C07K 1/14; C07K 14/245

(52) U.S. Cl. 435/69.7; 530/412

(57) ABSTRACT

The invention provides a method for the expression, purification, and structure recovery, of hydrophobic proteins, polypeptides or peptides in non-aggregated form, and for their convenient association with lipidic molecules. The method includes the steps of designing a fusion protein which contains in its amino acid sequence a hexa-His tag, and subsequently expressing the fusion protein using a vector in such a way as to target the expressed protein to bacterial inclusion bodies. The fusion protein also includes the desired hydrophobic protein, polypeptide or peptide and cyanogen bromide cleavage site or sites. The method also teaches isolation of the inclusion bodies, subsequent cleavage of the fusion protein, and purification of the recombinant protein, polypeptide or peptide using Nickel-chelate chromatography. The method also teaches recovery of the hydrophobic protein, polypeptide or peptide in unaggregated form with natural conformation and secondary structure and optimal association of the hydrophobic protein, polypeptide or peptide with lipidic molecules by dissolution in acidic organic solvents.

### FIELD OF THE INVENTION

The present invention relates to an improved method for the expression and purification of hydrophobic peptides and more particularly to an improved method for the expression, purification and structure recovery and handling of hydrophobic proteins, polypeptides or peptides.

### BACKGROUND OF THE INVENTION

Hydrophobic proteins, polypeptides and peptides are difficult to express in bacteria, possibly due to the fact that as bacteria manufacture hydrophobic proteins, polypeptides and peptides, the peptides end up in the host cell membranes which may lead to host weakness or death. Hereinafter the term “peptide” will be taken to include species commonly thought of as polypeptides and to include the term proteins.

If any quantity of hydrophobic peptides are manufactured it is generally difficult to purify them for several reasons including insolubility, excessive and unpredictable sticking to HPLC column packings, and the common dependence of conventional purification approaches on non-denaturing solvents and /or enzymatic cleavage. If purification is achieved, the peptides are often intractable due to insolubility and the fact that they may be denatured and/or aggregated.

Potential applications of hydrophobic peptides include, but are not limited to, their use in drug forms and in vaccine formulation. To-date, the lack of success in manufacturing, purifying and handling hydrophobic peptides have represented limitations to vaccine formulation since many important antigens are membrane proteins and hence have important distinguishing hydrophobic epitopes. In these applications it is also of great importance to achieve efficient presentation of protein epitopes to the host immune system, and therefore successful structure recovery and avoidance of protein aggregation are desirable.

Methods are known that teach the purification of hydrophobic peptides. One example is described in Canadian Application (CA)2,136,734 to Takei et al., which teaches a Purification Method for Hydrophobic Polypeptides. The method of CA 2,136,734 teaches the purification of hydrophobic polypeptides via High Performance Liquid Chromatography (HPLC) by using a mixed solvent containing a portion of trifluoroacetic acid. It is known in the art that HPLC methods of purifiying hydrophobic proteins and peptides often lead to unacceptable losses of product, small quantities of product, and impure product.

Another example of a purification method is described in CA 2,125,467 to Döbeli, H et al., which teaches a Process for Producing Hydrophobic Polypeptides, Proteins or Peptides. The method teaches binding the hydrophobic product non-specifically via hydrophobic attraction to a hydrophobic column for purification. The method teaches cleavage of the interim fusion protein product while bound to the hydrophobic column.

The method does not teach targeting of the desired fusion protein to inclusion bodies: inclusion bodies minimize proteolytic degradation, which is an important problem in commercial peptide preparation. Targeting to inclusion bodies also greatly simplifies purification of the product, which is another major consideration in commercial or academic work. The binding of the hydrophobic product to the hydrophobic column in the method of CA 2,125,467 risks contamination with other hydrophobic species formed during expression and cleavage.

The above mentioned reference CA 2,125,467 does not teach the recovery of the secondary structure of the peptides, proteins or polypeptides, or their dispersal as independent molecules, or their convenient and efficient association with a lipidic matrix.

The present invention teaches an important alternative purification method, and also deals with synthesis, structure optimization, and, where needed, transfer to lipidic matrices.

### SUMMARY OF THE INVENTION

It is therefore desirable to provide a method for expression, purification, and structure recovery and handling of hydrophobic peptides.

The method includes the steps of designing a fusion protein which contains in its amino acid sequence a hexa-His tag, and subsequently expressing the fusion protein using a vector in such a way as to target expressed peptides to inclusion bodies. The inclusion bodies are then isolated and undergo cleavage of the fusion protein to release the desired product and at the same time to expose the hexa-His tag. The recombinant peptides are then purified using Nickel-chelate chromatography. Recovery of the natural conformation and secondary structure of the peptides and their dispersion in un-aggregated form occurs by dissolution of the peptides in an acidic organic solvent that is compatible with common lipids.

### BRIEF DESCRIPTION OF THE DRAWINGS

The invention will be better understood with reference to the attached detailed description and to the following Figure, wherein

FIG. 1 is a ²H NMR spectra of deuterated peptides in fluid POPC bilayers at 35° C.

### DETAILED DESCRIPTION OF THE INVENTION

In a preferred embodiment of the invention, a method is provided for the expression, purification, and structure recovery and handling of hydrophobic peptides. The method includes the steps of constructing a vector coding for a fusion protein which contains within its amino acid sequence a protein or peptide that targets the fusion protein to bacterial inclusion bodies, a hexa-His tag, the desired hydrophobic peptide, and either a stop codon(s) contained within the DNA sequence coding for the fusion protein and/or cyanogen bromide cleavage sites in the fusion protein itself; subsequently expressing the fusion protein in such a way as to target expressed peptides to inclusion bodies. The method further includes the steps that the inclusion bodies are then isolated and the fusion protein is cleaved; the recombinant peptides are then purified using Nickel-chelate chromatography; and natural conformation and secondary structure of the peptides are recovered by dissolution of the peptides in an acidic organic solvent.

The fusion protein can be constructed in any desired sequence. For example, the hexa-His tag may be positioned immediately upstream or downstream of the desired hydrophobic peptide sequence. Also the hydrophobic peptide may be placed upstream or downstream of the protein that targets to inclusion bodies. However, the hexa-His tag must be directly attached to the hydrophobic peptide sequence of interest. Moreover the fusion protein preferably contains within its amino acid sequence beginning at the N-terminus a protein or peptide that targets the fusion protein to bacterial inclusion bodies, a cyanogen bromide cleavage site, a hexa-His tag, the desired hydrophobic peptide, and either a stop codon contained within the DNA sequence corresponding to the fusion protein or a further cyanogen bromide cleavage site in the fusion protein itself.

In the method of the present invention, when the desired hydrophobic peptide sequence contains a methionine residue(s) anywhere but where eventual cyanogen bromide cleavage is desired, the methionine residue(s) is/are either removed or substituted, by known methods of vector insert DNA design and construction, before expression of the fusion protein.

In a preferred embodiment of the method the peptide is expressed as a TrpE fusion protein in a pATH vector to target expression products to inclusion bodies, and subsequently the TrpE portion is removed by cyanogen bromide cleavage at the methionine residue between the TrpE protein and the hexa-His tag. This overcomes the common problem of low expressivity of hydrophobic peptides. Other vectors may also be used to target expression products to inclusion bodies, such as a pET vector. The method teaches cleavage of the interim fusion protein product before colurn purification. This approach permits easy and cheap use of small volumes of the toxic material employed to cleave the fusion protein. It also permits the toxic cleavage to be carried out in a small sealed, easily confined space, and also permits easy heating of the cleavage mixture to speed production. Inclusion bodies offer the additional benefits of reduced proteolytic degradation and simplified rapid purification. The presence of a hexa-His tag allows excellent recovery of the final peptide in very pure form, while permitting use of denaturing solvents and avoiding the need for HPLC with its attendant low capacity, poor separation of products, and adsorption losses.

The acidic organic solvent preferably comprises an acid that is capable of dissolving the peptide and an organic solvent that mimics the hydrocarbon interior of the membrane. The acid can be either a mineral acid or more preferably an organic acid, and examples of acids that may be used include formic acid, acetic acid and trifluoroacetic acid. The acidic organic solvent may also comprise an alcohol to assist in solubilizing the peptides, examples of alcohols that may be used include methanols, propanols and butanols. The preferred acidic solvent used in the above method comprises formic acid (90%), glacial acetic acid, chloroform and ethanol or trifluoroethanol, and preferably in the ratio 1:1:2:1. The acidic solvent is used in the recovery of secondary structure of the peptides, for the dispersion of product in non-aggregated form with natural conformation and secondary structure, and for optimal association with lipids.

The method of the present invention is described in Jones D. H. et al. Expression and Membrane Assembly of a Transmembrane Region from Neu, Biochemistry 2000, 39, 1870-1878 and in Sharpe, S. et al. Val⁶⁵⁹→Glu Mutation within the Transmembrane Domain of ErbB-2: Effects Measured by ²H NMR in Fluid Phospholipid Bilayers, Biochemistry 2000, 39, 6572-6580, incorporated herein by reference. All references referred to in the description are hereby incorporated by reference.

The method of the invention is further explained with reference to the following examples 1-6. The following sources were used for the material in the examples: 1-palmitoyl-2-oleoyl-3-sn-phosphatidylcholine (POPC) was obtained from Avanti Polar Lipids (Birmingham, Ala.) and was used without further purification. Deuterium-depleted water and deuteromethyl L-alanine were from Cambridge Isotope Laboratories (Andover, Mass.). 2,2,2-trifluoroethanol, NMR grade, bp 77-80EC was from Aldrich (Milwaukee, Wis.). Empigen (n-dodecyl-N,N-dimethylglycine) was from Calbiochem (San Diego, Calif.). Peptides made by solid phase synthesis were produced as described in Rigby, A. C. et al., (1996) Biochemistry 35, 12591-12601 and Jones, D. H. et al. (1998) Biochim. Biophys. Acta 1371, 199-212. Low molecular weight standards for SDS PAGE were bacitracin, insulin b chain, aprotinin, ∀-lactalbumin, myoglobin and triosephosphate isomerase (Bio-Rad, CA).

Definitions

Neu: a class I receptor tyrosine kinase of rat origin;

Neuₑₓₚ: a 50-residue expressed transmembrane peptide corresponding to Ala⁶⁴⁹ to Met⁶⁹² of Neu, with a hexa-His tag at the N-terminus;

Neu_{syn} a 38-residue synthetic transmembrane peptide corresponding to Pro⁶⁵⁵ to Thr⁶⁹¹ of Neu, with a Lys residue at the N terminus;

EGF: epidermal growth factor; ErbB-1, a human class I receptor tyrosine kinase (the Human EGF receptor);

ErbB-1_{syn}: a 34-residue synthetic transmembrane peptide corresponding to Ile⁶²² to Thr⁶⁵⁴ of ErbB-1;

ErbB-2ₑₓₚ: a 50-residue expressed transmembrane peptide comparable to Neuₑₓₚ but corresponding to Ala⁶⁴⁸ to Met⁶⁹¹ of the human protein homologous to Neu;

POPC: 1-palmitoyl-2-oleoyl phosphatidylcholine;

FACE, formic acid/acetic acid/chloroform ethanol {1:1:2:1 ratio by volume}; and

FACT, FACE with trifluoroethanol replacing ethanol.

### EXAMPLE 1

Construction of the TrpE-Neu Chimeric Protein.

A chimeric protein of anthranilate synthase, with the desired transmembrane sequence from the Neu proto-oncogene fused to the C-terminal end, was generated as follows. General techniques for molecular biology were performed according to standard protocols, for example as described in Sambrook, J. et al. (1989) Molecular cloning: a laboratory manual, 2^{nd} ed., Cold Spring Harbor Laboratory Press, Plainview, N.Y. The plasmid, pSV2neuN, as described in Bargmann, C. I. et al. (1986) Cell 45, 649-657, encoding the cDNA of the Neu proto-oncogene was kindly supplied by Dr. R. Weinberg (Whitehead Institute for Biomedical Research, Cambridge, Mass.). PCR was used to amplify the desired segment of the Neu sequence: the recombinant protein was initiated at position Ala⁶⁴⁹ and included the putative transmembrane domain. The upstream PCR oligonucleotide was GGAATTCCATATGCACCACCACCACCACCACGCAGAGCAGAGAGCCA (Seq.IDNo:1), which includes an EcoRI site (GAATTC), an NdeI site (CATATG), a hexa-His tag, and the initial amino acids of the Neu sequence. The downstream oligonucleotide primer used was CATGCCATGGCCTCCACTAACTCAGTTTCCT (Seq.IDNo:2), which runs to Glu⁷⁰³ in Neu and includes sites for NcoI and PstI. Since the PCR products proved difficult to digest they were ligated into a pGEM-T-vector (Promega). After verification by sequencing, it was double digested with EcoRI and PstI and the fragment isolated from a 2% SeaPlaque GTG agarose (FMC) gel according to the manufacturer=s instructions. This fragment was then ligated into the pATH11 vector, for example as described in Koerner, T. J. et al. (1991) Methods Enzymol. 194, 477-490, which had been similarly digested and purified. The construct was confirmed by sequencing, and designated “pATH-Neu”. All cloning steps were performed in E. coli strain JM101, although other suitable strains or bacterium may also be used.

### EXAMPLE 2

Expression of the TrpE-Neu Fusion Protein.

Recombinant proteins were expressed following the method as described in Koemner et al., other suitable methods may also be employed. Several different strains of E. coli were tested for expression of the chimeric protein, including JM101, BL21, DH5α, and JM109. Cells were grown to an OD₆₀₀ of 0.4 in M9 media supplemented with 5 mg/ml Bacto-casamino acids (Difco), 1 mM MgSO₄, 0.1 mM CaCl₂, 0.2% glucose, 0.01 mg/ml thiamin B1, 0.02 mg/ml tryptophan and 100 μg/ml carbenicillin (Sigma). This culture was diluted 10 fold into the same media lacking Trp and incubated at 37EC for two hours. Indoleacrylic acid (Sigma) was then added to a final concentration of 9 μg/ml and the suspension incubated for an additional 4 hours. In order to achieve alanine labelling the bacteria were>force fed=deuterated alanine: the casamino acids were omitted and the M9 media was supplemented with all amino acids (including deuterated alanine), except for Trp, at a concentration of 100-200 μg/ml, as described in Muchmore, D. C. et al. (1989) Methods Enzymol. 177, 44-73, other methods of alanine labelling may also be used.

### EXAMPLE 3

Isolation of the Expressed Neu Peptide, Neuₑₓₚ.

TrpE fusion proteins, including TrpE-Neu of the present work, target to inclusion bodies, which were isolated in the following manner. Cells were harvested, resuspended in 3 ml Lysis buffer containing 0.15 mM PMSF per gram cell pellet, as described in Sambrook, J. et al., subjected to two French press cycles, and centrifuged at 12,000×g. Pelleted inclusion bodies were washed twice with lysis buffer containing 0.1% TritonX-100 and total protein was determined using the DC Protein Assay (Bio-Rad). The pellet was then treated with cyanogen bromide (CNBr), which cleaves at Met residues, to separate the peptide, Neuₑₓₚ, from the fusion protein. For this purpose the pellet was dissolved in 20 ml of 70% formic acid, and 3 mg of CNBr was added per mg total protein. The mixture was incubated at 42EC for 4 hours, and then lyophilised after adding an equal volume of water. The resultant dry powder was suspended in 20 ml of 9% Empigen, as described in Sanders II, C. R. et al. (1996) Biochemistry 35, 8610-8618, and re-lyophilised to remove any residual CNBr. This step was repeated with 20 ml of water.

### EXAMPLE 4

Purification of Neu Recombinant Peptide.

The peptide, Neuₑₓₚ, (with attached hexa-His tag) was purified by nickel-chelate chromatography, as described in Janknecht, R. et al. (1991) Proc. Natl. Acad. Sci. U.S.A. 88, 8972-8976, after cleavage from the fusion protein as above. The CNBr cleavage products were dissolved in 6 M GuHCl, 0.1 M NaH₂PO₄, 0.01 M Tris-Cl, pH 8.0 over a period of up to 6 hr with periodic brief bath sonication. The resultant solution was centrifuged at low speed to remove particulate matter and then incubated overnight with nickel-NTA resin (Qiagen) at room temperature on a rocker table. The quantity of resin used was according to the manufacturer=s recommended binding capacity, assuming that all the isolated protein corresponded to the protein of interest. After incubation, the resin was loaded onto a 0.9×15 cm Pharmacia column and washed with 10 bed volumes of 6 M GuHCl, 0.1 M NaH₂PO₄, 0.01 M Tris-Cl, pH 8.0. This was followed by a series of 8 M urea, 0.1 M NaH₂PO₄, 0.01 M Tris-Cl washes in which the pH was progressively lowered in steps from 8.0 to 6.3, 5.9, and 4.5. At each step the column was washed with 10 bed volumes of each solution. The Neuₑₓₚ peptide was finally eluted by washing the column with 10 bed volumes of 6 M guanidine hydrochloride containing 0.2 M acetic acid. Yield of pure peptide was typically 4-5 mg per litre of growth medium: the only significant product by mass spectroscopy had the expected target Mᵣ. Peaks corresponding to dimers or higher oligomers were not found by mass spectrometry.

### EXAMPLE 5

Gel Electrophoresis and Western Blots.

SDS PAGE was performed using a mini-gel system (Bio-Rad). Larger proteins were typically run on 12% gels as described by Laemmli, U. K. (1970) Nature 227, 680-685. Small proteins were run on the 16.5% tris-tricine gels described by Schagger H. (1994) in A practical guide to membrane protein purification (Schagger, H. and Von Jagow, G., Eds.) Chapter 3, Academic Press, San Diego. Samples were dissolved in standard loading buffer, as described in Sambrook J. et al., and incubated for 30 min at 42EC prior to loading. Gels were stained with Coomassie Brilliant Blue. Following gel transfer to a PVDF membrane [Gelman Sciences] Western blots were performed to detect the hexa-His tag using a HisProbe Western Blotting kit (Pierce). This kit utilises Ni-activated horseradish peroxidase to detect polyhistidine proteins. The manufacturer=s procedure was followed except that blots were blocked overnight with 4% casein in TBST. Samples for electrophoresis were usually pretreated with organic acid. Such pretreatment typically involved dissolution and incubation for 1 hour in formic acid (90%)/glacial acetic acid/chloroform/ethanol (1:1:2:1 ratio by volume)(AFACE@ described in Findlay, J. B. C. (1990) in Protein purification applications, a practical approach (Harris, E. L. V., and Angal, S., Eds.) IRL Press, Oxford) or in the same solvent with ethanol replaced by trifluoroethanol (AFACT@), prior to drying under a stream of nitrogen followed by vacuum desiccation for 16 hr. Samples were heated in loading buffer for 30 minutes at 42EC prior to loading onto the gel.

### EXAMPLE 6

Preparation of Samples for NMR Spectroscopy.

Except where noted otherwise, liposome generation was according to the following protocol. Acidic organic solvent mixtures, particularly FACT, were employed to prepare solutions of lipid plus peptide that could be dried to form thin films for subsequent hydration with sample buffer. Typically 4 ml solvent was added to dry peptide (10 mg) and appropriate amounts of dry lipid, with warming to 45EC. Samples were incubated at this temperature for at least 30 min after visually-apparent complete dissolution. Solvent was then rapidly removed under reduced pressure at 45EC on a rotary evaporator to leave thin films in 50 ml round bottom flasks. These were subsequently vacuum desiccated for 18 h at 23EC under high vacuum with continuous evacuation. Even prolonged exposure to the acid solvents used did not give rise to detectable lipid degradation as measured on heavily-overloaded thin layer chromatography plates. Hydration was with 30 mlM HEPES with 20 mM NaCl and 5 mM EDTA pH 7.1-7.3, made up in deuterium-depleted water (vortexing was avoided to minimize production of small vesicles). ²H NMR spectra were acquired at 76.7 MHz on a Varian Unity 500 spectrometer using a single-tuned Doty 5 mm solenoid probe with temperature regulation to ∀ 0.1 CE. A quadrupolar echo sequence, described in Davis, J. H. (1991) in Isotopes in Physical and Biomedical Science (Buncel, E. and Jones, J. R. Eds.) Vol. 2, Elsevier, Amsterdam, (“SSECHO” from the Varian pulse library) was employed with full phase cycling and B/2 pulse length of 5-6 μs. Pulse spacing was typically 20 μs, and sweep width was 100 kHz.

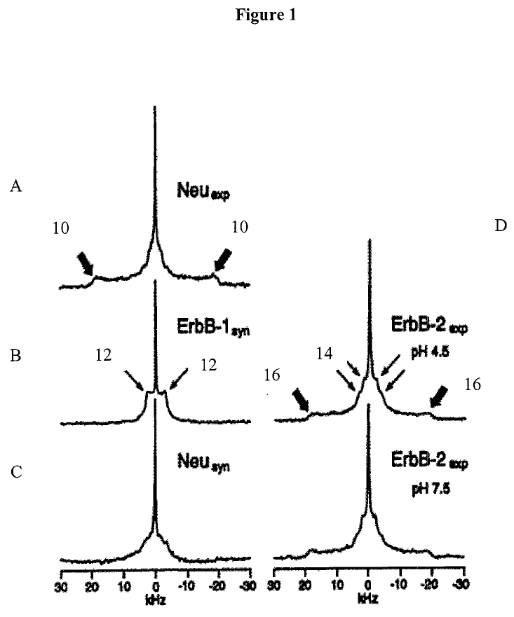

FIG. 1 shows the ²H NMR spectra of dueterated peptides in fluid POPC bilayers at 35° C. Each peptide contained one or more alanine residues with perdeuterated side chains (−CD₃). Key spectral features referred to are indicated by arrows. Peptide/phospholipid mol ratio was 6 mol %. The number of accumulated transients represented by each spectrum ranged from 100,000 to 1,300,000. Expressed peptide spectra have been normalised to constant area. The top spectrum A corresponds to the expressed 50-mer, Neuₑₓₚ, deuterated at all 3 alanine sites. Below it for comparison are typical spectra of the shorter (synthetic) peptides, ErbB-1_{syn} (B) deuterated at Ala⁶²³ and Neu_{syn} (C) dueterated at Ala⁶⁶¹. Thick arrows 10 in spectra of Neuₑₓₚ indicate the limiting 40 kHz splitting expected of alanines in peptides that are immobilized by lateral associations. Thin arrows 12 in the case of ErbB-1_{syn} indicate the narrowed Pake doublet characteristic of rapid symmetrical peptide rotation. In the right column D shows spectra of the 50-amino-acid expressed transmembrane peptide, ErbB-2ₑₓₚ, for samples in which the hexa-His tag carries a charge of +6 (upper) and zero (lower). Thin arrows 14 in the upper ErbB-2exp spectrum indicate the two narrowed Pake doublets (from the two deuterated alanines) associated with a rapidly-rotating fraction of ErbB-2ₑₓₚ; while the thick arrows 16 indicate wider features associated with peptide molecules involved in longer term lateral associations.

While the embodiment discussed herein is directed to a particular implementation of the invention, it will be apparent that variations of this embodiment are within the scope of the invention. For example, the vector can be any vector that directs the proteins expressed in the bacterium to inclusion bodies. Similarly, the fusion protein can be any fusion protein whose corresponding DNA sequence can be used in an expression vector to direct the proteins to inclusion bodies. The acidic organic solvent used in the process can be any acidic organic solvent that is capable of dissolving the peptide and has hydrocarbon-like properties.

We claim:

1. A method for expression, purification and structural recovery of hydrophobic proteins, polypeptides or peptides comprising the steps of:
i) constructing a vector coding for a fusion protein which contains within its amino acid sequence a protein or peptide that targets the fusion protein to bacterial inclusion bodies, a hexa-His tag, the desired hydrophobic protein, polypeptide, or peptide, and at least one of at least one stop codon contained within the DNA sequence coding for the fusion protein or at least one cyanogen bromide cleavage site in the fusion protein itself;
ii) expressing the fusion protein using a vector to target the expressed fusion protein to inclusion bodies;
iii) isolating the inclusion bodies and cleavage of the fusion protein;
iv) purifying the recombinant protein, polypeptide or peptide using Nickel-chelate chromatography; and
v) recovering natural conformation and secondary structure of the protein, polypeptide or peptide by dissolution in an acidic organic solvent, and at the same time assuring an unaggregated form
wherein when the desired hydrophobic protein, polypeptide or peptide contains a methionine residue in a location where one does not wish cleavage by cyanogen bromide, the methionine residue is either removed or substituted by manipulation of the coding DNA during design of the expression vector for the fusion protein.

2. The method as claimed in claim 1, wherein the fusion protein is a TrpE fusion protein.

3. The method as claimed in claim 2, wherein the fusion protein is expressed in a bacterium.

4. The method as claimed in claim 4, wherein the bacterium is E.coli.

5. The method as claimed in claim 5, wherein the bacterium is selected from the group comprising the strains JM101, BL21, DH5∀, and JM109.

6. The method as claimed in claim 1, wherein the vector is a pATH vector or a pET vector.

7. The method as claimed in claim 7, wherein the vector is a pATH11 vector.

8. The method as claimed in claim 1, wherein the acidic organic solvent comprises a mixture of an acid that can dissolve the protein, polypeptide or peptide and an organic solvent.

9. The method as claimed in claim 9, wherein the acidic organic solvent comprises a mixture of (a) formic acid, glacial acetic acid, chloroform and ethanol, or (b) formic acid, glacial acetic acid, chloroform and trifluoroethanol.

10. The method as claimed in claim 10, wherein the acidic organic solvent mixture of (a) and (b) is in a 1:1:2:1 ratio.

11. A method for expression and purification of hydrophobic proteins, polypeptides or peptides comprising the steps of:
i) expressing a fusion protein which contains within its amino acid sequence a protein or peptide that targets the fusion protein to bacterial inclusion bodies, a cyanogen bromide cleavage site comprising a methionine residue, a hexa-His tag, and the desired hydrophobic protein, polypeptide or peptide followed by either a stop codon contained within the DNA sequence coding for the fusion protein or a further cyanogen bromide cleavage site comprising a methionine residue;
ii) expressing the fusion protein using a vector to target the expressed fusion protein to inclusion bodies;
iii) isolating the inclusion bodies and cleavage of the fusion protein; and
iv) purifying the recombinant proteins, polypeptides or peptides using Nickel-chelate chromatography
wherein when the hydrophobic protein, polypeptide or peptide contains a methionine residue, the methionine residue is either removed or substituted by standard manipulation of the coding DNA before expression of the fusion protein.

12. The method as claimed in claim 11, wherein the vector is a pATH vector or a pET vector.

13. An acidic organic solvent comprising a mixture of formic acid, glacial acetic acid, chloroform and trifluoroethanol.

14. An acidic organic solvent as claimed in claim 13, wherein the formic acid, glacial acetic acid, chloroform and trifluoroethanol exist in a 1:1:2:1 ratio.

15. The use of an acidic organic solvent to assure that the final peptide is not aggregated and for recovery of secondary structure of hydrophobic proteins, polypeptides or peptides.

16. The use as claimed in claim 15, wherein the acidic organic solvent comprises a mixture of an acid that can dissolve the proteins, polypeptides or peptides and an organic solvent.

17. The use as claimed in claim 16, wherein the acidic organic solvent comprises a mixture of formic acid, glacial acetic acid, chloroform and ethanol or trifluoroethanol.

18. The use of the acidic organic solvent of claim 16 for the assembly of hydrophobic peptides into lipid bilayers or other lipidic phase.
